(19) United States
(12) Patent Application Publication
(10) Pub. No.: US 2011/0230914 A1
(43) Pub. Date: Sep. 22, 2011

(54) DYNAMIC CABLE SYSTEM

(75) Inventors: Volker Engelmann, Muenchenstein (CH); Markus Kraft, Frenkendorf (CH); Beat Lechmann, Grenchen (CH)

(73) Assignee: SYNTHES (U.S.A.), West Chester, PA (US)

(21) Appl. No.: 12/672,051

(22) PCT Filed: Aug. 7, 2008
(86) PCT No.: PCT/US2008/072481

§ 371 Date: Feb. 3, 2010

Related U.S. Application Data
(60) Provisional application No. 60/954,443, filed on Aug. 7, 2007.

(51) Int. Cl. A61B 17/70

(52) U.S. Cl. 606/263; 606/264

(57) ABSTRACT

A dynamic cable system (11) for spanning two or more adjacent vertebrae V includes a longitudinal cable (12) having an inner cavity (12a) and at least one damping material (13) disposed within the inner cavity. Each of the vertebrae includes at least one bone fixation element (10) attached thereto. The bone fixation elements include a channel formed therein. The longitudinal cable is positionable within the channel and the longitudinal cable is a plait cable, a woven cable, a braided cable, a knitted cable, a twisted cable or a tube. The dynamic fixation system can include a first bone fixation element mounted to the first vertebra, a second bone fixation element mounted to the second vertebra, a first clamping sleeve (19) including a first bore (33), a second clamping sleeve including a second bore, the longitudinal cable having a first end, a second end, and an inner cavity, and the damping material disposed at least within the inner cavity.

### CROSS-REFERENCE TO RELATED APPLICATIONS

This application claims the benefit of U.S. Provisional Application No. 60/954,443, filed on Aug. 7, 2007, titled “DYNAMIC CABLE SYSTEM,” the contents of which is incorporated in its entirety by reference herein.

### BACKGROUND OF THE INVENTION

Spinal fusion is a procedure that involves joining two or more adjacent vertebrae to restrict movement of the vertebra with respect to one another. For a number of known reasons, spinal fixation devices are used in spine surgery to align and/or secure a desired relationship between adjacent vertebral bodies. Such devices typically include a spinal fixation element, such as a relatively rigid fixation rod, that is coupled to adjacent vertebrae by attaching the fixation element to various bone fixation elements, such as hooks, bolts, wires, screws, etc. The fixation elements can have a predetermined contour, and once installed, the fixation element holds the vertebrae in a desired spatial relationship, either until desired healing or spinal fusion has taken place, or for some longer period of time.

Dynamic fixation elements are desirable at least in part because they absorb shock, for example, in the extension and compression of the spine. In addition, the removal of bone structure, such as facet joints or laminae, result in instabilities of the motion segments of the spine. Consequently, a fixation system should stabilize the motion segment in antero-posterior translation as well as in axial rotation. Both motion patterns result in shear stress within the spinal fixation element of the fixation system. This is especially important in elderly patients, where the bone quality is sometimes compromised, becoming sclerotic or osteoporotic.

It is desirable to have a dynamic fixation system that provides constraints regarding shear stresses and improves stabilization without limiting the system's range of motion in flexion. It is also desirable to provide a system comprising a low number of components to reduce the complexity of the assembly.

### BRIEF SUMMARY OF THE INVENTION

The preferred embodiment of the present invention is directed to a dynamic cable system for posterior spinal fixation. The dynamic cable system is preferably sized and configured to span two or more adjacent vertebrae, each of the vertebrae having at least one bone fixation element attached thereto. The bone fixation elements each include a channel formed therein.

In one exemplary embodiment, the dynamic cable system includes a longitudinal cable having an inner cavity. The longitudinal cable is preferably sized and configured to be received within the channel formed in the bone fixation elements and at least one damping material is disposed within the inner cavity of the cable. The cable is preferably in the form of a plait, woven, braided, knitted or twisted cable. Alternatively, the cable may be in the form of a tube, preferably a twisted tube.

The damping material preferably may be injection molded into the inner cavity of the cable. More preferably, the damping material may be injection molded into the inner cavity of the cable through gaps formed in the plait, woven, braided, knitted or twisted cable or tube. Additionally and/or alternatively, the dynamic cable system may include damping material injection molded around the cable so that at least a portion of the cable is encapsulated by the damping material.

The dynamic cable system may also include at least one clamping sleeve. The clamping sleeve preferably may include a bore to receive, preferably slidably receive, at least a portion of the cable. The clamping sleeve is preferably received within the channel formed in the bone fixation element so that the clamping sleeve can be disposed within the channel formed in the bone fixation element and the cable can be disposed within the bore formed in the clamping sleeve. The portion of the cable that is received within the bore is preferably devoid of any damping material. The clamping sleeve may also include a plurality of tabs extending from an end thereof, wherein the tabs are separated by recesses.

The dynamic cable system preferably includes at least two adjacent clamping sleeves and damping material disposed around, at least partially, the adjacent clamping sleeves and the portion of the cable disposed therebetween.

In another exemplary embodiment, the dynamic cable system includes at least two clamping sleeves, wherein each of the clamping sleeves includes a bore. The clamping sleeves are preferably received within the channel formed in the bone fixation elements. The dynamic cable system may also include a longitudinal cable having a first end, a second end, and an inner cavity. The first end of the longitudinal cable is preferably received within the bore formed in one of the clamping sleeves. The second end of the longitudinal cable is preferably received within the bore formed in the other clamping sleeve. At least one damping material is disposed within the inner cavity of the cable. The cable is preferably in the form of a plait, woven, braided, knitted or twisted cable. Alternatively, the cable may be in the form of a tube, preferably a twisted tube.

### BRIEF DESCRIPTION OF THE SEVERAL VIEWS OF THE DRAWINGS

The foregoing summary, as well as the following detailed description of a preferred embodiment of the application, will be better understood when read in conjunction with the appended drawings. For the purposes of illustrating the device of the present application, there is shown in the drawings a preferred embodiment. It should be understood, however, that the application is not limited to the precise arrangements and instrumentalities shown. In the drawings:

FIG. 1 is a side view of a spine segment showing an exemplary embodiment of the dynamic cable system secured thereto;

FIG. 2 is a cross-sectional view of the exemplary embodiment of the dynamic cable system of FIG. 1;

FIG. 3 is a cross-sectional view of the exemplary embodiment of the dynamic cable system of FIG. 2 under tension;

FIG. 4 is a cross-sectional view of the exemplary embodiment of the dynamic cable system of FIG. 2 under compression;

FIG. 5 is a cross-sectional view of the exemplary embodiment of the dynamic cable system of FIG. 2 incorporating optional clamping sleeves;

FIG. 6 is a perspective view of the dynamic cable system incorporating optional clamping sleeves of FIG. 5;

FIG. 7 is a cross-sectional view of the dynamic cable system incorporating optional clamping sleeves of FIG. 6; and

FIG. 8 is another perspective view of an exemplary embodiment of the dynamic cable system incorporating optional clamping sleeves.

### DETAILED DESCRIPTION OF THE INVENTION

Certain terminology is used in the following description for convenience only and is not limiting. The words “right”, “left”, “lower” and “upper” designate directions in the drawings to which reference is made. The words “inwardly” and “outwardly” refer to directions toward and away from, respectively, the geometric center of the device and designated parts thereof. The words, “anterior”, “posterior”, “superior”, “inferior” and related words and/or phrases designate preferred positions and orientations in the human body to which reference is made and are not meant to be limiting. The terminology includes the above-listed words, derivatives thereof and words of similar import.

An exemplary embodiment of the invention will now be described with reference to the drawings. In general, such embodiments relate to a fixation system, by way of non-limiting example, a dynamic fixation system for posterior spinal fixation. As will be described in greater detail below, the dynamic fixation system may comprise a dynamic cable system including a longitudinal cable and/or cord, preferably a plait, woven, braided, knitted or twisted cable. Alternatively, the cable may be in the form of a tube, preferably a twisted tube, or other similar shape. However, it should be understood that other forms and/or shapes are envisioned. The plait cable or cord, woven cable or cord, braided cable or cord, knitted cable or cord, twisted cable or cord, tubular cable and/or twisted cable shall be referred to herein as a cable, however, it should be understood that the terms may be used interchangeably. The dynamic cable system may also include a damping material and/or component (collectively referred to herein as a damping material). The damping material may be injection molded into the cable. Alternatively and/or additionally, the damping material may be injection molded around and/or over the cable. The dynamic cable system may also incorporate one or more clamping sleeves.

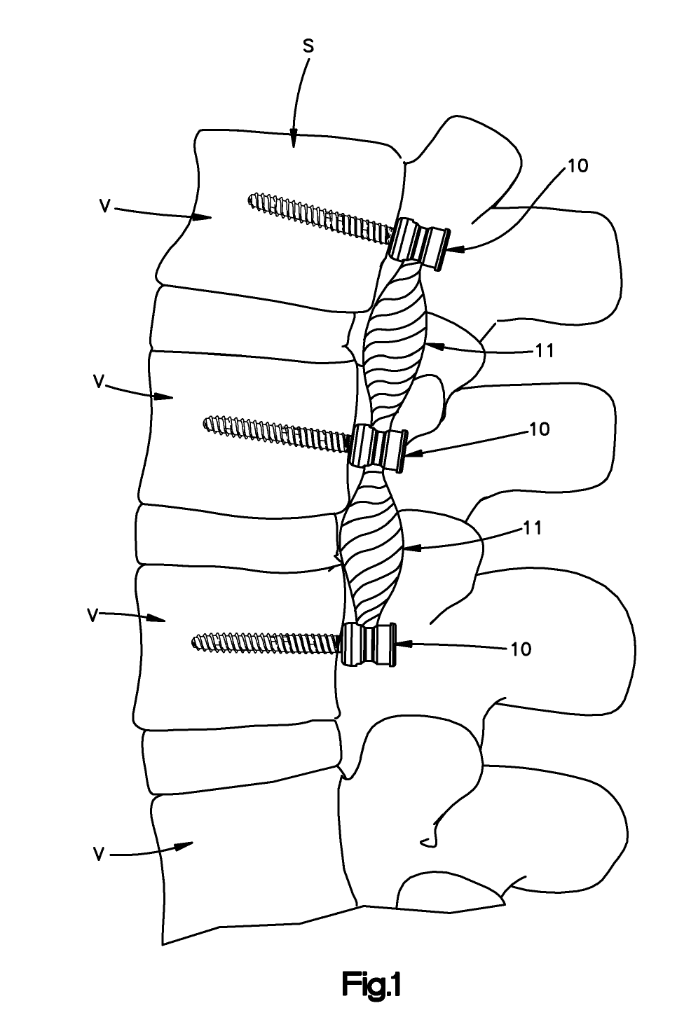

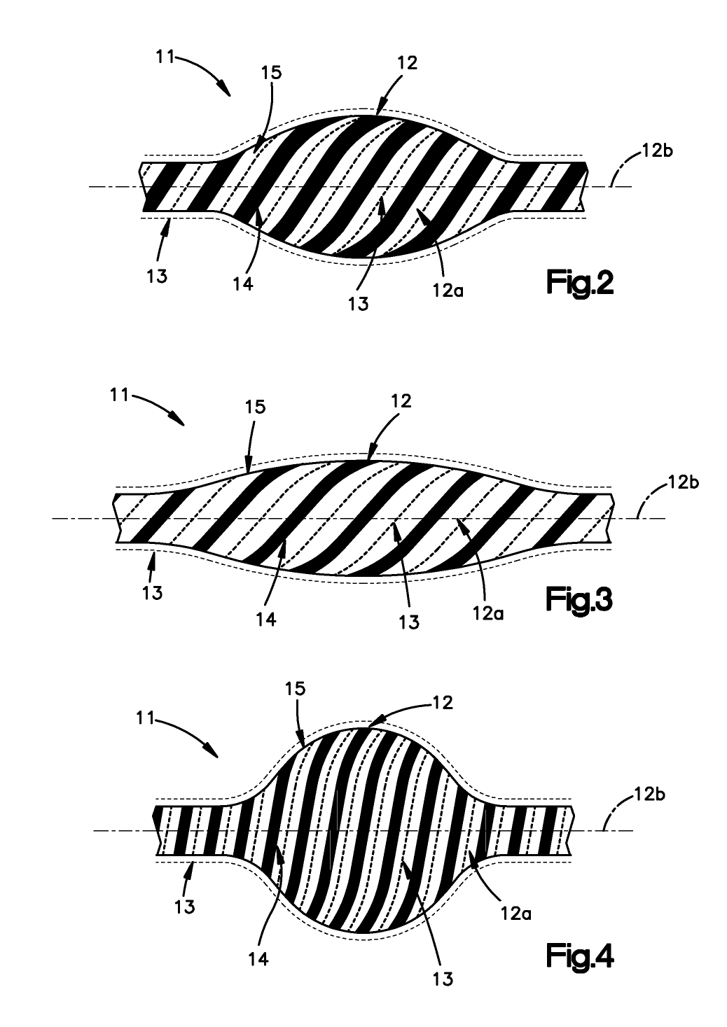

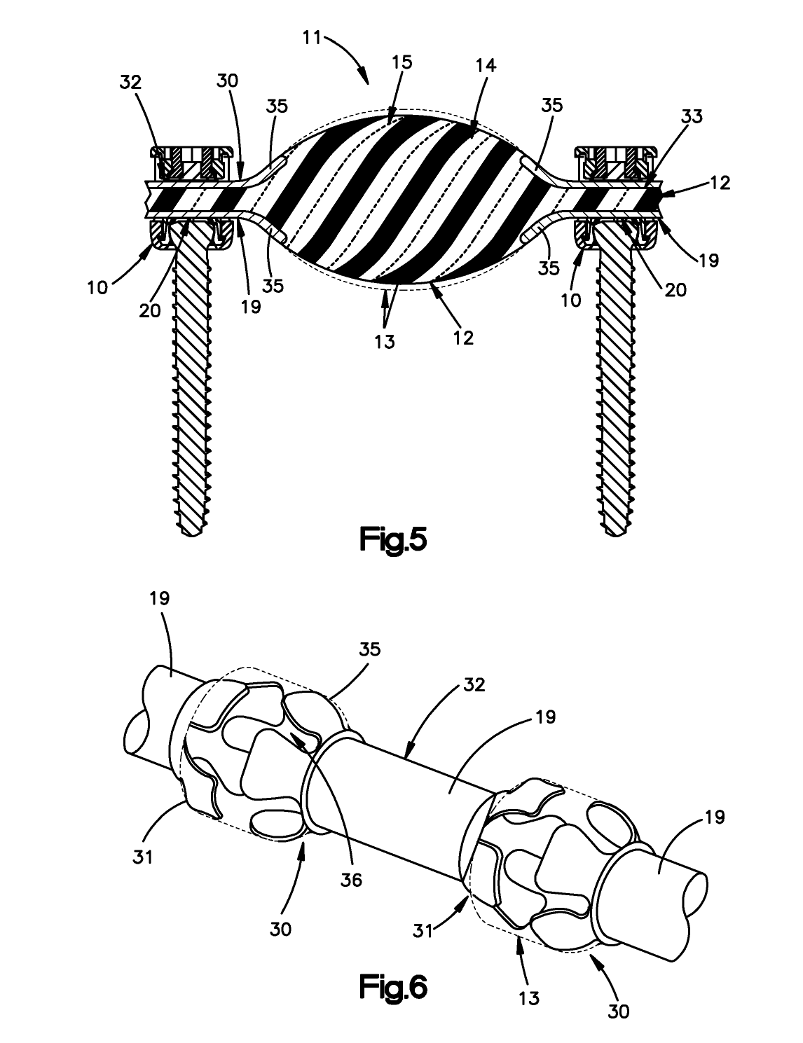

Referring to FIGS. 1-8, a bone fixation element, generally indicated as 10, includes, but not limited to, poly-axial or mono-axial pedicle screws, hooks (both mono-axial and poly-axial) including pedicle hooks, transverse process hooks, sublaminar hook, or other fasteners, clamps or implants, and in no way is a dynamic cable system 11 of the present application limited to use with any particular type of bone fixation element 10.

A cable 12 of the preferred embodiment of the present application is manufactured from any biocompatible material known in the art including, but not limited to, members of the poly aryl ether ketone family, for example, poly ether ether ketone (PEEK), poly ether ketone ketone (PEKK), poly ether ketone (PEK), etc., member of the polyester family, for example, poly ethylene terephthalate (PET), poly butyl terephthalate (PBT), etc., polyethylene fibers, ultra high molecular weight polyethylene (UHMWPE), glass fibers, cobalt chromium, carbon fibers, aramid fibers, stainless steel, plastics, carbon fiber reinforced matrices, carbon fiber reinforced plastics, etc. Preferably, the cable 12 is manufactured from titanium or titanium alloys.

A damping material 13 of the preferred embodiment of the present application may be made of, for example, a gel core, a hydrogel, a silicone, elastomeric components and/or materials, a rubber, a thermoplastic elastomer, or a combination thereof. Preferably, the damping material 13 is constructed of polycarbonate urethane (PCU). The elasticity of the damping material is preferably higher than that of the remaining components of the dynamic cable system 11 including the cable 12 and optional clamping sleeves.

Clamping sleeves 19 of the preferred embodiment of the present application are constructed of any biocompatible material known in the art including, but not limited to, members of the aryl ether ketone family, for example, poly ether ether ketone (PEEK), poly ether ketone ketone (PEKK), poly ether ketone (PEK), etc., members of the polyester family, for example, poly ethylene terephthalate (PET), poly butyl terephthalate (PBT), etc., polyethylene fibers, glass fibers, cobalt chromium, titanium, titanium alloys, carbon fibers, aramid fibers, stainless steel, plastics, carbon fiber reinforced matrices, carbon fiber reinforced plastic, etc.

In situ, the dynamic cable system 11 may engage one or more bone fixation elements 10, which engage one or more vertebrae V so that the dynamic cable system 11 spans two or more adjacent vertebrae V for stabilization (e.g., stabilizing or fixation) of the vertebrae V with respect to one another. For example, the dynamic cable system 11 may be used in combination with an intervertebral implant (not shown). The dynamic cable system 11 may permit the vertebrae V to settle (e.g. compress) over time, thus facilitating fusion between the intervertebral implant and the adjacent vertebrae V. Alternatively, the dynamic cable system 11 may be used in connection with an articulating intervertebral implant (not shown) or any other implant known in the art, or none at all. Moreover, the amount and type of movement that the dynamic cable system 11 can be tailored for individual patients. For example, for patients with less severe pathologies (e.g., better bone structure), a less stiff system may be desirable to permit additional movement. Likewise, for patients with more degenerate disks, a stiffer system may be desirable to permit less or no movement.

As generally understood by one of ordinary skill in the art, the dynamic cable system 11 may be used to span adjacent vertebrae V. Alternatively, any number of vertebrae V may be spanned by the dynamic cable system 11. For example, the dynamic cable system 11 may be used to span three or more vertebrae V.

Moreover, while the dynamic cable system 11 will be described as and may generally be used in the spine S (for example, in the lumbar, thoracic and/or cervical regions), those skilled in the art will appreciate that the dynamic cable system 11, as well as the components thereof, may be used for fixation of other parts of the body such as, for example, joints, long bones or bones in the hand, face, feet, etc.

Referring to FIG. 1, the individual vertebrae V may be stabilized posteriorly. Specifically, the bone fixation elements 10 are secured into three vertebrae V from the posterior direction. Heads of the bone fixation elements 10 each have a channel, commonly referred to as a rod-receiving channel, for accommodating and/or receiving portions of the dynamic cable system 11, respectively. The dynamic cable system 11 is preferably capable of being fixed with respect to the bone fixation elements 10 by securing the dynamic cable system 11 in the channels by, for example, a closure cap or set screw, as generally understood by one of ordinary skill in the art. In this manner, the spine S of the patient can be stabilized.

Referring to FIGS. 1-4, the dynamic cable system 11 includes the longitudinal cable 12, which incorporates an inner cavity 12a. The damping material 13 is preferably disposed within the inner cavity 12a to provide damping characteristics to the cable 12. The cable 12 is preferably manufactured from individual strands and/or fibers 14 (collectively referred to herein as fibers) which are braided together. The damping material 13 may be inserted into the cable 12 so that the damping material 13 may be, at least partially, surrounded by, or at least partially encapsulated by, the cable 12. This may be achieved, for example, by twisting the cable 12 so that the individual fibers 14 of the cable 12 separate so that gaps 15 develop between the individual fibers 14. The damping material 13 is preferably inserted into the inner cavity 12a via the gaps 15. Alternatively, the cable 12 may include naturally occurring gaps 15 between the individual fibers 14, which may obviate the need to twist the fibers 14 apart. Alternatively, the damping material 13 may be inserted into the inner cavity 12a formed in the cable 12 by any means known in the art. For example, the damping material 13 may be pre-molded into any number of shapes, for example, a cylinder or ovoid, and subsequently inserted into the inner cavity 12a of the cable 12.

The damping material 13 is preferably injection molded into the inner cavity 12a of the cable 12, more preferably in-between the gaps 15 formed between the individual fibers 14 of the cable 12. In this manner, as the damping material 13 cures and hardens, the damping material 13 may fill the gaps 15, which in turn may assist in keeping the damping material 13 from disengaging and/or separating from the cable 12.

Alternatively and/or additionally, the damping material 13 may be injection molded around the cable 12 so that the damping material 13 at least partially surrounds the cable 12. In this manner the damping material 13 occupies the space formed by the inner cavity 12a of the cable 12, the space formed by the gaps 15 in-between the individual fibers 14, and at least partially surrounds the cable 12. Different damping materials with differing elastomeric qualities may be used to construct the damping material 13. For example, the damping material 13 may be constructed of a first material inserted into the inner cavity 12a of the cable 12 and a second material surrounding the cable 12. In the preferred embodiment, the damping material 13 is constructed of the same material.

In situ, as the attached vertebrae V move, the movement and associated loads are transferred from the vertebrae V to the dynamic cable system 11, via the bone fixation elements 10. In this manner, the dynamic cable system 11 permits the attached vertebrae V to move with respect to one another. The combination of the flexible cable 12 and the damping material 13 may absorb some or all of the movement (e.g., translation, articulation, rotational (e.g., twisting), etc.) and associated loads and/or stresses.

Referring to FIG. 3, when the dynamic cable system 11 is loaded in tension, the cable 12 lengthens, resulting in a narrowing of the cable 12 at its midsection. The tension/flexion stresses may be absorbed, at least partially by the cable 12 and the lateral compression stresses may be transferred to and absorbed, at least partially, by the damping material 13. The use of the cable 12 may also limit distortion of the damping material 13 by limiting the axial and translational movement of the damping material 13.

The amount of permitted axial movement of the cable 12 may be constrained, for example, by the angle at which the individual fibers 14 of the cable 12 are wrapped about a longitudinal axis 12b of the cable 12. For example, wrapping the fibers 14 at a flatter angle (i.e., more parallel to the longitudinal axis 12b) allows for less axial movement than wrapping the fibers 14 at a steeper angle (i.e., more perpendicular to the longitudinal axis 12b). Preferably, the fibers 14 of the cable 12 may be wrapped at an angle ranging from about fifteen degrees (15°) to about seventy-five degrees (75°). More preferably, the fibers 14 may be wrapped at an angle ranging from about twenty-five degrees (25°) to about sixty-five degrees (65°). More preferably, the fibers 14 may be wrapped at an angle of about forty-five degrees (45°).

The amount of permitted rotational movement of the dynamic cable system 11 may be constrained, for example, by manufacturing the cable 12 from two or more sets of fibers 14, which may be braided in opposite directions. In one embodiment, for example, two sets of fibers 14 may be intermeshed. Additionally and/or alternatively, one set of fibers 14 may be wrapped around another set of fibers 14. Each set of fibers 14 may limit rotational movement in the direction in which the fibers 14 are braided, such as, for example, similar to the braided fibers used in tires or carbon reinforced fiber matrices. In addition, two or more coaxially braided fibers or cords can be used in order to achieve a gradual increase in stiffness (as a function of distance f(x) from the longitudinal axis 12b). The fibers 14 used may be twisted, braided, woven or knitted.

Referring to FIG. 4, loading the dynamic cable system 11 under compression results in the cable 12 compressing and/or coiling, which in turn results in a widening of the cable 12 at its midsection. Consequently, the axial compression stress may be transferred to and absorbed, at least partially, by the damping material 13.

Referring to FIGS. 5-7, the dynamic cable system 11 may be used in conjunction with one or more clamping sleeves 19. The clamping sleeves 19 may include a first end 30, a second end 31, an intermediate clamping area 32, and a bore 33 extending from the first end 30 to the second end 31. The intermediate clamping area 32 is preferably received in, and subsequently secured in, the channel formed in the bone fixation element 10. Alternatively, the clamping sleeves 19 may only include a first end 30, a clamping area 32 and a bore 33. This configuration may be particularly useful for spanning two adjacent vertebrae V or for use on the end vertebra V when spanning three of more vertebrae V.

The clamping sleeves 19 preferably surround, at least partially, the dynamic cable system 11 and more preferably the cable 12. That is, the bore 33 formed in the clamping sleeve 19 preferably receives the cable 12 therein. The cable 12 is preferably slidably disposed within the bore 33 of the clamping sleeve 19. The clamping sleeves 19 preferably surround the cable 12 in the location where the cable 12 is received within the channels of the bone fixation elements 10 (e.g., clamping site 20). The clamping sleeves 19 facilitate attachment of the dynamic cable system 11 to the bone fixation elements 10, which in turn are secured to the vertebrae V. The clamping sleeves 19 preferably surround the cable 12 in order to protect the cable 12 from shearing at the clamping sites 20. Thus, the clamping sleeves 19 protect the cable 12 against plastic deformation and notch stresses caused by the bone fixation elements 10 as the cable 12 undergoes compression and tension.

First and second ends 30, 31 of the clamping sleeves 19 may include a plurality of tabs 35 extending therefrom that are separated by a plurality of recesses 36. The tabs 35 and recesses 36 preferably enable a gradual decrease in stiffness such as, for example, when the dampening element 13 exhibits increased deformations, as may be the case during translation and/or flexion/extension. As will be generally appreciated, the clamping sleeves 19 preferably allow for deformation in the clamping site 20 while still protecting the damping material 13 from notch stresses. Moreover, the tabs 35 and recesses 36 formed on adjacent clamping sleeves 19 may be rotationally offset with respect to one another so that the tabs 35 formed on one sleeve 19 align with the recesses 36 formed on the adjacent sleeve 19. As shown, the clamping sleeves 19 may include four tabs 35, arranged uniformly around the first and second ends 30, 31 of the clamping sleeve 19, although it is envisioned that more or less tabs 35 may be used.

The damping material 13 is preferably injection molded into the cable 12 after the cable 12 has been inserted into the clamping sleeves 19. In this manner, little, if any, damping material 13 is located in the clamped section 32 of the cable 12 (e.g., the section of the cable 12 inserted into the bore 33 of the clamping sleeves 19). Alternatively, the entire cable 12 may include damping material 13 disposed therein. As previously mentioned, the cable 12 may be freely received and/or slidably disposed within the bore 33 of the clamping sleeve 19. The cable 12 may alternatively be secured to the clamping sleeves 19. Preferably, the cable 12 is freely received and/or slidably disposed within the bore 33 of the clamping sleeve 19 up until the damping material 13 is injection molded into the inner cavity 12a of the cable 12. After injection molding, the position of the cable 12 is preferably fixed with respect to the clamping sleeve 19. The cable 12 may be secured to the clamping sleeves 19 by any means known in the art including, but not limited to, adhesive, crimping of the clamping sleeves 19, screws, bolts, clamps, pins, braided, etc.

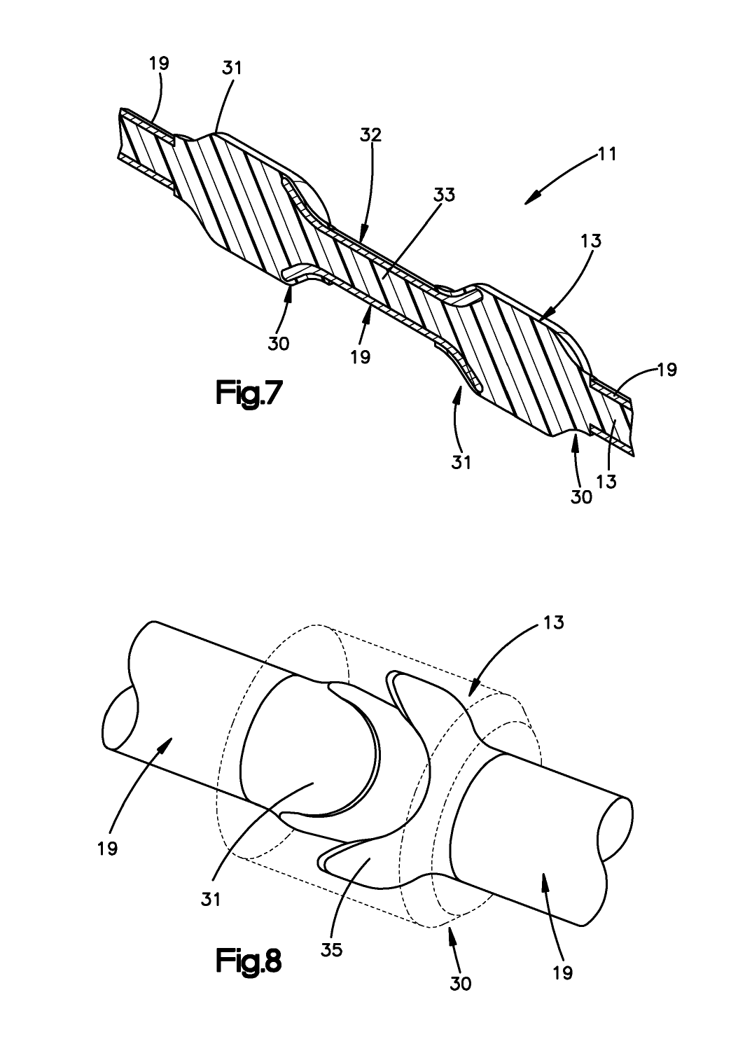

Referring to FIGS. 7 and 8, the dynamic cable system 11 may also incorporate additional damping material 13 molded or injection molded around the cable 12. The additional damping material 13 preferably surrounds and/or encases, at least partially, the first and second ends 30, 31 of adjacent clamping sleeves 19 and preferably the exposed segment of the cable 12. The additional damping material 13 may absorb some of the shear forces, as well as some of the shock from extension or compression of the spine S. In addition, incorporating additional damping material 13 around the exposed segment of the cable 12 may help to ensure that the entirety of the cable 12 is protected against wear and accumulation of debris. That is, the optional additional damping material 13 disposed around the cable 12 may be seen as a protective layer preventing wear debris from escaping from the dynamic cable system 11.

In use, the length of the dynamic cable system 11 will depend on the size and number of vertebrae V being secured. For example, the length of the cable 12 may be up to one meter (1 m) long, if the patient's entire spine is being secured and/or instrumented. As will be generally understood by one of ordinary skill in the art, the diameter of the cable 12 will be sized to absorb the expected loads. Thus, the cable 12 sized for use in the lumbar region will typically have a larger diameter than are cable 12 sized for use in the thoracic or cervical region. For example, the diameter of the cable 12 may range from one millimeter (1 mm) to twenty millimeters (20 mm) for use with in the lumbar region of the spine, or from one millimeter (1 mm) to fifteen millimeters (15 mm) for use in the cervical region of the spine. Alternatively, the cable 12 may have a uniform diameter extending the entire length thereof. The thinner or clamped sections of the cable 12 may be manufactured by tightly twisting or braiding the cable 12 so that a thinner part for the clamping area is achieved.

As will be appreciated by those skilled in the art, any or all of the components described herein such as, for example, the bone fixation elements 10, the cable 12, the clamping sleeves 19, etc. may be provided in sets or kits so that the surgeon may select various combinations of components to perform a fixation procedure and create a fixation system which is configured specifically for the particular needs/anatomy of a patient. It should be noted that one or more of each component may be provided in a kit or set. In some kits or sets, the same device may be provided in different shapes and/or sizes (e.g., multiple bone fixation elements 10, cables 12 and/or clamping sleeves 19 of different sizes).

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

What is claimed is:

1. A dynamic cable system for spanning two or more adjacent vertebrae, each of the vertebrae having at least one bone fixation element attached thereto, the bone fixation elements each including a channel formed therein, the dynamic cable system comprising:
a longitudinal cable having an inner cavity, the longitudinal cable positionable within the channels; and
at least one damping material injection molded into the inner cavity, wherein the longitudinal cable is selected from the group consisting of a plait cable, a woven cable, a braided cable, a knitted cable, a twisted cable and a tube.

2. The dynamic cable system of claim 1, wherein the cable includes a plurality of fibers separated by a plurality of gaps, the at least one damping material is injection molded into the inner cavity of the cable through the gaps formed in the cable.

3. The dynamic cable system of claim 1, wherein the at least one damping material is also injection molded around the cable so that at least a portion of the cable is encapsulated by the damping material.

4. The dynamic cable system of claim 1, further comprising:
at least one clamping sleeve including a bore that receives at least a portion of the cable, the at least one clamping sleeve received within one of the channels.

5. The dynamic cable system of claim 4, wherein the at least a portion of the cable received in the bore is devoid of any damping material.

6. The dynamic cable system of claim 4, wherein the at least one clamping sleeve includes a first end and a second end, the first end includes a plurality of tabs separated by recesses.

7. The dynamic cable system of claim 4, wherein the at least one clamping sleeve includes first, second and third clamping sleeves, the first, second and third clamping sleeves each having a first end, a second end and an intermediate clamping area, the first, second and third clamping sleeves arranged coaxial with a longitudinal axis of the longitudinal cable, which extends through the bores of the first, second and third clamping sleeves, first and second ends of the second clamping sleeve facing the first clamping sleeve and the second clamping sleeve, respectively, the at least one damping material disposed around, at least partially, the first, second and third clamping sleeves and the longitudinal cable.

8. The dynamic cable system of claim 1, wherein the cable includes two or more sets of fibers that are braided, knitted or twisted together in opposite directions.

9. A dynamic fixation system for spanning a first vertebra and a second vertebra, the first vertebra positioned adjacent to the second vertebra, the dynamic fixation system comprising:
a first bone fixation element mounted to the first vertebra, the first bone fixation element having a first channel formed therein;
a second bone fixation element mounted to the second vertebra, the second bone fixation element having a second channel formed therein;
a first clamping sleeve including a first bore, the first clamping sleeve positioned at least partially within the first channel;
a second clamping sleeve including a second bore, the second clamping sleeve positioned at least partially within the second channel;
a longitudinal cable having a first end, a second end, and an inner cavity, the first end received within the first bore and the second end received within the second bore; and
a damping material disposed at least within the inner cavity, wherein the cable is selected from one of a plait cable, a woven cable, a braided cable, a knitted cable, a twisted cable, or a tube.

10. The dynamic fixation system of claim 9, wherein the at least one damping material is injection molded into the inner cavity.

11. The dynamic fixation system of claim 10, wherein the longitudinal cable includes a plurality of fibers separated by a plurality of gaps, the damping material being injection molded into the inner cavity of the longitudinal cable through the plurality of gaps.

12. The dynamic fixation system of claim 11, further comprising:
at least one damping material injection molded around the longitudinal cable so that at least a portion of the longitudinal cable is encapsulated by the damping material.

13. The dynamic fixation system of claim 12, wherein the damping material is injection molded around the cable and at least a portion of the clamping sleeves.

14. The dynamic fixation system of claim 9, wherein the first and second ends of the longitudinal cable are devoid of the damping material.

15. The dynamic fixation system of claim 9, wherein the first and second clamping sleeves include a plurality of tabs separated by recesses.
